(19) United States
(12) Patent Application Publication
(10) Pub. No.: US 2021/0335069 A1
(43) Pub. Date: Oct. 28, 2021

(54) HOSPITAL ACCESS CONTROL SYSTEM AND HOSPITAL ACCESS CONTROL METHOD UTILIZING SAME

(71) Applicants: Gi Yul AHN, Gwangju (KR); G-orlin Corp., Jeonju-si (KR)

(72) Inventor: Gi Yul AHN, Gwangju (KR)

(21) Appl. No.: 17/159,876

(22) Filed: Jan. 27, 2021

(30) Foreign Application Priority Data
Apr. 22, 2020 (KR) 10-2020-0048491

(51) Int. Cl. G07C 9/00

(52) U.S. Cl. CPC G07C 9/00896; G07C 9/00182; G07C 2009/00277; G07C 2009/00269; G07C 9/00563

(57) ABSTRACT

Disclosed herein are a hospital access control system and a hospital access control method utilizing the same, which can classify users according to different access areas according to personal information, provide access authentication means for the users, and determine whether or not the access areas applied to the access authentication means are authorized access areas in order to manage entrance and exit points, floors, spaces, traffic lines and the likes for the users thoroughly and rigorously, thereby constructing a rigorous infection prevention management system in the hospital that unspecified individuals use, and minimizing damages in the hospital by actively coping with infections in case of issues of infections.

### CROSS-REFERENCES TO RELATED APPLICATIONS

This application claims priority to and the benefit of Korean Patent Application No. 10-2020-0048491, filed on Apr. 22, 2020, the disclosure of which is incorporated herein by reference in its entirety.

### BACKGROUND OF THE INVENTION

### Field of the Invention

The present invention relates to a hospital access control system and a hospital access control method utilizing the same, and more particularly, to a hospital access control system and a hospital access control method utilizing the same, which can classify users who enter a hospital, for instance, medical service persons, cooperative firm workers, patients, protectors, visitors, and others, by different access areas according to personal information, provide access authentication means for the users, and determine whether or not the access areas applied to the access authentication means are authorized access areas in order to manage entrance and exit points, floors, spaces, traffic lines and the likes for the users thoroughly and rigorously, thereby constructing a rigorous infection prevention management system in the hospital that unspecified individuals use, and minimizing damages in the hospital by actively coping with infections in case of issues of infections.

### Background Art

Recently, respiratory infection diseases, such as SARS, MERSC, and COVID-19, are prevalent. As such respiratory infection diseases quickly spread to the whole building by infected persons who visit large buildings, such as large-scaled hospitals, and sanitariums, people take measures like cohort isolation.

In case of hospitals, because most of users are people with weaker immune systems, if they are exposed to respiratory infection diseases, it may cause serious mass infection by movement of infectious agents, and may cause death of the people with deteriorated immune systems.

Especially, most of hospitals are not equipped with proper access control systems for controlling entrance and exist of visitors. Even though the hospitals are equipped with access control systems, because most of gates of the hospitals are opened at ordinary times and anyone can enter and exit hospitals during visiting hours, it is difficult to previously block mass infection by unspecified individuals.

Furthermore, now, visitors who visit a hospital must write their visiting schedules and entry logs on a daily record. However, because of frequent omission of records, if mass infection occurs, epidemiological survey is virtually impossible.

Therefore, a systematic and accurate infection prevention management system which is applicable to hospitals to guarantee a more secure access control is needed.

### PATENT LITERATURE

### Patent Documents

Patent Document 1: Korean Patent No. 10-2073823

### SUMMARY OF THE INVENTION

Accordingly, the present invention has been made to solve the above-mentioned problems occurring in the prior arts, and it is an object of the present invention to provide a hospital access control system and a hospital access control method utilizing the same, which can classify users by different access areas according to personal information, provide access authentication means for the users, and determine whether or not the access areas applied to the access authentication means are authorized access areas in order to manage entrance and exit points, floors, spaces, traffic lines and the likes for the users thoroughly and rigorously, thereby constructing a rigorous infection prevention management system in the hospital that unspecified individuals use, and minimizing damages in the hospital by actively coping with infections in case of issues of infections.

Especially, the present invention provides a hospital access control system and a hospital access control method utilizing the same, which can limit usable gates, floors, spaces and traffic lines by access areas authorized to users, and thoroughly control use time.

It is another object of the present invention to provide a hospital access control system and a hospital access control method utilizing the same, which can be linked with a firefighting interworking system constrFFlucted in a hospital in case of fire so as to quickly and forcibly open all gates in the hospital.

It is a further object of the present invention to provide a hospital access control system and a hospital access control method utilizing the same, which can be linked with an electronic medical record system (EMR) or an order communication system (OCS) constructed in the hospital.

To accomplish the above object, according to the present invention, there is provided a hospital access control system including: a visitor authentication unit for confirming hospital users and obtaining personal information; an access authentication means provider which subdivides and classifies the users by predetermined access areas based on the personal information provided from the visitor authentication unit, and gives access authentication means allotted by the access areas to the classified users; and a gate control unit which is connected with each gate in the hospital and with an access authentication means reader disposed at each gate, determines whether the access area where the user scanned his or her access authentication means through a specific access authentication means reader is an authorized access area when the user scans his or her access authentication means, and determines whether to open the gate.

In an embodiment, the visitor authentication unit includes: a temperature measuring means for measuring temperature of the user using a thermal imaging camera or a temperature measuring sensor; and a personal information obtaining means for obtaining personal information from the user through a terminal.

In an embodiment, the access authentication means provider classifies hospital staff or cooperative firm workers to a first access area, patients and protectors to a second access area, visitors to a third access area, and other users to a fourth access area.

In an embodiment, the access authentication means include character codes including RF cards, barcodes or QR codes, and mobile cards, and biometric information, the access authentication means reader includes an RF card reader, a character code scanner, and biometric information scanner for recognizing biometric information, such as fingerprint, vein or iris, and the gate control unit checks whether the access area where the user scans his or her access authentication means corresponds to any one among the first to fourth access areas based on any one of the RF card, the character code and the biometric information scanned through the access authentication means reader.

In an embodiment, the gate control unit differentially applies locations and opening hours of gates, which are authorized to be opened, according to the access areas.

In an embodiment, the access authentication means further includes a database for automatically recording and storing the locations and scanning time of the access authentication means readers where the access authentication means is scanned.

In an embodiment, the gate control unit is linked with a firefighting system constructed in the hospital so as to forcibly open all gates in the hospital in case of fire.

In an embodiment, the gate control unit is connected with all elevators in the hospital to control buttons of the elevators to be capable of being manipulated if the access area where the access authentication means is scanned through the access authentication means reader in the elevator is the authorized access area.

In an embodiment, the hospital access control system is linked with an electronic medical record system (EMR) or an order communication system (OCS) constructed in the hospital.

In an embodiment, the gate control unit synchronizes each of the access authentication means readers mounted at each of the gates in the hospital and the present conditions of each access area in real time.

In another aspect of the present invention, the present invention provides a hospital access control method using a hospital access control system including the steps of: confirming hospital users through a visitor authentication unit and obtaining personal information; classifying the hospital users by predetermined access areas using an access authentication means provider, based on the personal information of each user obtained through the visitor authentication unit, and giving access authentication means allotted by the access areas to the classified users; and determining, through a gate control unit connected with each gate and an access authentication means reader disposed at each gate, whether the access area where the access authentication means is scanned is the authorized access area when the access authentication means is scanned through the access authentication means reader so as to decide whether to open the gate.

The hospital access control system and the hospital access control method utilizing the same according to an embodiment of the present invention can classify users by different access areas according to personal information, provide access authentication means for the users, and determine whether or not the access areas applied to the access authentication means are authorized access areas in order to manage entrance and exit points, floors, spaces, traffic lines and the likes for the users thoroughly and rigorously, thereby constructing a rigorous infection prevention management system in the hospital that unspecified individuals use, and minimizing damages in the hospital by actively coping with infections in case of issues of infections.

Especially, the hospital access control system and the hospital access control method utilizing the same according to an embodiment of the present invention can limit usable gates, floors, spaces and traffic lines by access areas authorized to users, and thoroughly control use time.

Moreover, the hospital access control system and the hospital access control method utilizing the same according to an embodiment of the present invention can be linked with a firefighting interworking system constructed in a hospital in case of fire so as to quickly and forcibly open all gates in the hospital, thereby initially coping with fire and preventing a mass mortality event by fire.

Furthermore, the hospital access control system and the hospital access control method utilizing the same according to an embodiment of the present invention can be linked with an electronic medical record system (EMR) or an order communication system (OCS) constructed in the hospital and can be linked with integrated access data based on a view table provided from the hospital, thereby adjusting table lookup time intervals as appropriate for environments.

Additionally, in consideration of cooling and heating environment of the hospital, the hospital access control system according to the present invention can control the gate to be automatically opened when visitors approach without any authentication in designated areas, and can control the gates in variable ways according to hospital environments, for instance, using contactless sensor type buttons for leaving the hospital.

In addition, the present invention can individually and forcibly open the gates in the hospital and control all of the gates except specific gates managed by infection prevention personnel, thereby thoroughly performing infection prevention management.

Moreover, the present invention can set freely and independently set access zones, days and time by entrances, gates, floors, elevators, users' traffic lines in the hospital, thereby minimizing damages in the hospital by actively coping with infection in case of an issue of infection.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a view showing a general process for entering a hospital via a screening center;

FIG. 2 is a schematic diagram showing a configuration of a hospital access control system according to a preferred embodiment of the present invention;

FIG. 3 is a flow chart showing a process of forcibly opening gates in a hospital in link with a firefighting system constructed in the hospital by a gate control unit illustrated in FIG. 2; and

FIG. 4 is a flow chart showing a method of controlling access of users in the hospital utilizing the hospital access control system according to the embodiment of the present invention.

### DETAILED DESCRIPTION OF THE PREFERRED EMBODIMENT

Hereinafter, a preferred embodiment of the present invention will be described in detail with reference to the accompanying drawings. However, the embodiment is provided only to help more easy understanding of the present invention, and the present invention is not limited to the embodiment.

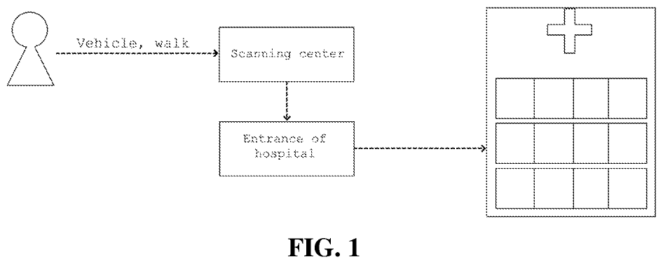

FIG. 1 is a view showing a general process for entering a hospital via a screening center.

Referring to FIG. 1, when respiratory diseases which are highly contagious are prevalent, persons who use a hospital go to a screening center after arrival at a parking lot in the hospital or walk to the screening center in the hospital.

During the process, there is a need to securely identify the hospital users at the entrance of the hospital and control accessible areas and possible traffic lines based on personal information because the users have no special symptom at the time of advance medical treatment in the screening center but may have some symptoms after several days.

Next, processes working at a gate after the authorization process carried out before the users who passed the screening center actually enter the hospital will be described in detail.

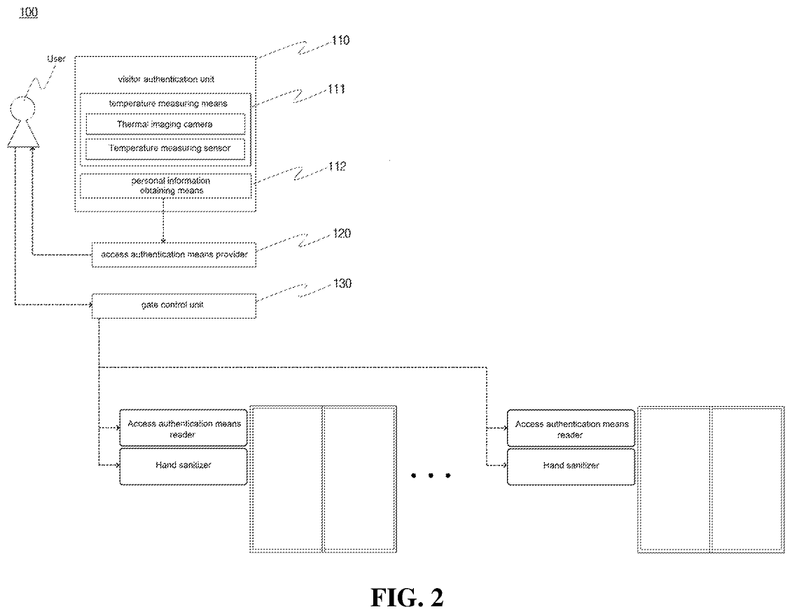

FIG. 2 is a schematic diagram showing a configuration of a hospital access control system 100 according to a preferred embodiment of the present invention.

Referring to FIG. 2, the hospital access control system 100 according to the embodiment of the present invention includes a visitor authentication unit 110, an access authentication means provider 120, and a gate control unit 130.

First, the visitor authentication unit 110 authorizes hospital users or visitors, obtains personal information, and provides the obtained personal information and the authorized users or visitors to the access authentication means provider 120.

The visitor authentication unit 110 includes a temperature measuring means 111 and a personal information obtaining means 112.

The temperature measuring means 111 may be a thermal imaging camera or a temperature measuring sensor, and the personal information obtaining means 112 may be an electronic terminal, which can record and store user's personal information, such as tablet PCs, notebooks, PDAs, desktop computers, kiosks or the likes. Moreover, the personal information obtaining means 112 can store not only the user's personal information but also visit records, such as visiting time, purposes, and others.

The personal information obtained through the personal information obtaining means 112 is provided to the access authentication means provider 120 which will be described later, and the access authentication means provider 120 subdivides and classifies the hospital users or visitors based on the information.

The access authentication means provider 120 subdivides and classifies the users according to predetermined access areas based on the personal information provided from the visitor authentication unit 110, and gives the classified users access authentication means allotted by the access areas.

In more detail, the hospital users mean hospital staff, workers for cooperative firms which are relevant with the hospital, patients, protectors, visitors, and other persons who have other business.

Therefore, the access authentication means provider 120 subdivides and classifies the people with various purposes by at least four access areas.

Here, to classify users by the predetermined access areas means to divide the users according to different levels. That is, because there are different levels authorized to hospital users, access authentication is applicable to the users differentially. The access areas may be understood as ‘security level’, ‘access level’, or ‘user level’.

For instance, hospital staff or cooperative firm workers are classified to a first access area, patients and protectors are classified to a second access area, visitors are classified to a third access area, and other users are classified to a fourth access area.

That is, the users for the first access area can use entrances, zones, floors, gates, spaces, and traffic lines in the hospital the widest, the user for the second access area are restricted more than the users for the first access area in use of the entrances, zones, floors, gates, spaces, and traffic lines in the hospital. In this instance, hospitals may have different restricted matters.

In the same manner, it is obvious that the users for the third access area are restricted more than the users for the first access area and the users for the fourth access area are restricted more than the users for the third access area.

The access authentication means provider 120 classifies the users to the first to fourth access areas in order to enhance security in the hospital from unspecified persons using the hospital and utilize the classified materials as epidemiologic survey data through a systematic management in case of emergency.

Meanwhile, when the access authentication means provider 120 provides access authentication means allotted to the access areas for the classified users, the access authentication means may be cards on which character codes are printed, such as contactless type RF cards, barcodes or QR codes, mobile cards outputted through a user's mobile terminal screen, or input of biometric information, such as face perception, iris recognition, fingerprint recognition, vein recognition, or palm vein recognition. In an embodiment, the cards on which character codes, such as bar codes or QR codes are printed may be in a sticker type.

That is, the access authentication means provider 120 can obtain biometric information from a user for the first access area, make and store a database of the information, and provide an RF card capable of recording and tracking the user.

Moreover, the databased and stored personal information may be selectively stored according to set periods, for instance, for one month, and may be set to be discarded automatically when the predetermined period elapses.

Furthermore, the access authentication means provider 120 may give the user for the second access area an RF card or a card on which character codes are printed, or give patients bracelets on which character codes are printed. Therefore, the users (patients or protectors) for the second access area can use designated places, floors, gates and spaces during appointed time. If the user for the second access area approach an access authentication means reader using the RF card, or the card or bracelet on which character codes are printed, gates are not opened because the access area is not allowed.

Additionally, the access authentication means provider 120 may give the user for the third access area an RF card only for visitors. In addition, the access authentication means provider 120 may give the user for the fourth access area an RF card only for the user. Likewise, also the users for the third and fourth access areas can use designated places, floors, gates and spaces during appointed time.

Meanwhile, when the access authentication means given to the users for the first to fourth access areas are scanned through the access authentication means reader, scanning positions, time and others are all recorded automatically and databased. Moreover, the databased and stored personal information may be selectively stored according to set periods, for instance, for one month, and may be set to be discarded automatically when the predetermined period elapses. The visitor authentication unit 110 having the temperature measuring means 111 and the personal information obtaining means 112, and the access authentication means provider 120 are mounted in an integrated information guide system (kiosk), so that the kiosk can be operated in an unmanned manner, and can rapidly handle access procedures if users visit the hospital after reservation through a website or an application.

Furthermore, if a CCTV or an emergency bell is installed to be linked with the system, in case of an emergency, the system can rapidly notify a safety guard or a manager of the situation through a control room or a mobile device so that the safety guard or the manager can cope with the situation rapidly.

Next, the gate control unit 130 is connected with gates, speed gates, sensor gates, mobile control devices, and access authentication means readers disposed on elevators in the hospital, decides whether an access area is the authorized access area when an access authentication means is scanned through the access authentication means reader at the access area, and controls opening and closing of the gate after determining whether to open the gate for the corresponding user based on the decision.

In more detail, each of the gate control units 130 are connected with each of the access authentication means readers mounted on all gates and all elevators existing in the hospital so as to control the gates and elevators through interworking with the access authentication means readers.

Here, the access authentication means reader means a device which can scan the access authentication means, such as RF cards, cards or bracelets on which character codes are printed, biometric information or others.

For instance, the access authentication means readers may be RF card readers, character code scanners which can scan character codes, fingerprint recognition scanners, face recognition cameras or scanners, vein recognition scanners, or iris recognition scanners.

Therefore, the gate control unit 130 determines the user's access area relative to the access authentication means to be scanned on the basis of data obtained through the access authentication means reader.

For instance, entrances, floors, spaces, traffic lines, movement radiuses, and time of the first access area may be authorized through the access authentication means given to the users for the first access area, the users for the first access area can freely move within the range of the corresponding space and time through his or her access authentication means. Scanning results of the access authentication means through the access authentication means reader, and location and scanning time of the access authentication means reader are recorded and stored in the database automatically.

Additionally, entrances, floors, spaces, traffic lines, movement radiuses, and time of the second access area, which are restricted more than those of the first access area may be authorized through the access authentication means given to the users for the second access area, the users for the second access area can freely move within the range of the corresponding space and time through his or her access authentication means. Likewise, scanning results of the access authentication means through the access authentication means reader, and location and scanning time of the access authentication means reader are recorded and stored in the database automatically. Descriptions of the third access area and the fourth access area will be omitted since they are the same as the first and second access areas.

That is, the gate control unit 130 differentiates locations and opening time of gates which can be opened according to the access areas allotted by the access authentication means. In this instance, locations and opening time of gates which can be opened according to the first to fourth access areas can be changed through a manager terminal.

In the meantime, in another embodiment, the gate control unit 130 is connected not only with the access authentication means readers mounted on the gates and elevators in the hospital but also disinfecting devices, such as spray-type hand sanitizers, or air shower booths. The gate control unit 130 determines whether the access area where the access authentication means is scanned through the access authentication means reader is the authorized access area, and determines and controls opening and closing of the gate for the corresponding user only when the user completes the disinfection process through the disinfecting device.

In more detail, each of the gate control units 130 is connected with each of the access authentication means readers and each of disinfecting devices mounted on all gates and elevators in the hospital to control the gates and the elevators while interworking with the access authentication means readers and disinfecting devices.

For instance, in case of an intensive care unit requiring disinfection essentially, the corresponding gate is opened only when it is determined that disinfection is completed by a disinfecting device even after access authentication is performed through the access authentication means reader.

In this instance, in order to determine completion of disinfection, the number of times that disinfectant is sprayed after the user holds out his or her hand toward the disinfecting device is counted, or a procedure to check whether the disinfecting device carries out disinfection is performed.

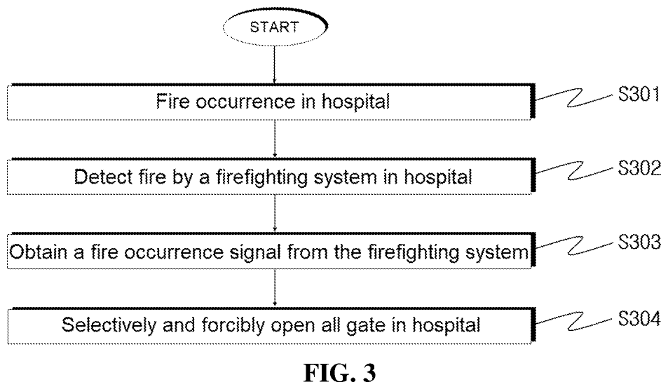

Moreover, the gate control unit 130 is linked with a firefighting system constructed in the hospital in order to cope with fire by forcibly opening all gates in the hospital in case of fire. Referring to FIG. 3, the process of firefighting will be described.

FIG. 3 is a flow chart showing a process of forcibly opening gates in the hospital in link with the firefighting system constructed in the hospital by the gate control unit illustrated in FIG. 2.

Referring to FIG. 3, in case of fire (S301), the firefighting system constructed in the hospital detects it (S302), the gate control unit 130 according to the present invention obtains a signal of fire occurrence from the firefighting system (S303), and forcibly opens all gates connected with the gate control units 130 (S304). In this instance, the gate control unit 130 may control the gates predetermined to be forcibly opened selectively or all at once so as to forcibly open the gates. Furthermore, the gate control unit 130 can block an electric locking device in a physical switching manner so that users can manually open the gates even though the system is not operated normally due to power disconnection.

Referring to FIG. 2, the gate control unit 130 is connected with the elevator in the hospital, determines whether the access area where the access authentication means is scanned through the access authentication means reader mounted on the elevator is the authorized access area, and controls buttons in the elevator to be manipulated by a user if the access area is the authorized access area.

For instance, if the floors that the users for the first access area can use are from the first floor to the tenth floor and the floors that the users for the second access area can use are from the first floor to the fifth floor, when the user for the second access area scans his or her access authentication means through the access authentication means reader, an alarm or a message informing that only buttons corresponding to the first floor to the fifth floor are capable of being manipulated may be outputted, or the buttons corresponding to the first floor to the fifth floor may be activated. Besides the above, button manipulation of the elevator may be differentially applied to the users corresponding to the first to fourth access areas in various ways.

In an embodiment, the gate control unit 130 can synchronize each of the access authentication means readers mounted at each of the gates in the hospital and the present conditions of each access area in real time so as to automatically open the gate within a specific time period.

For instance, doctors, medical staff and cooperative firm workers corresponding to the first access area and patients and protectors corresponding to the second access area can enter the gate in real time when they scan their access authentication means within three minutes if they enter the same gate, but leavers, discharged patients, and protectors for discharged patients cannot access through their access authentication means. It can be realized through interworking with an electronic medical record system (EMR) or an order communication system (OCS) constructed in the hospital.

In an embodiment, the gate control unit 130 can set visiting time by days of week differentially to visitors corresponding to the third access area.

Moreover, in consideration of cooling and heating environment of the hospital, the gate control unit 130 can control the gate to be automatically opened when visitors approach without scanning in designated areas within possible visiting hours if the gate has an automatic door.

Meanwhile, the hospital access control system 100 according to the present invention can obtain a worker data table from the OCS constructed in the hospital, can automatically record 48-hour working conditions of workers in the hospital based on the worker data table, and can be linked with access control systems of wards and departments, a parking control system, a meal quantity management system, an education management system, an RF card admission ticket output system, and a sticker type barcode output system.

In the meantime, the hospital access control system 100 according to the present invention can selectively apply some of the processes through a manager terminal if a manager determines that it is necessary to actively cope with infection of contagious diseases in case of an issue of infection of a contagious disease.

For instance, at ordinary days when there is no issue of infection of a contagious disease, the hospital access control system 100 according to the present invention performs only the temperature measuring process and the process of obtaining personal information through the temperature measuring means and the personal information obtaining means. At abnormal days when there is an unexpected issue of infection of a contagious disease, the hospital access control system 100 according to the present invention additionally performs the processes of classifying users by access areas and providing the access authentication means.

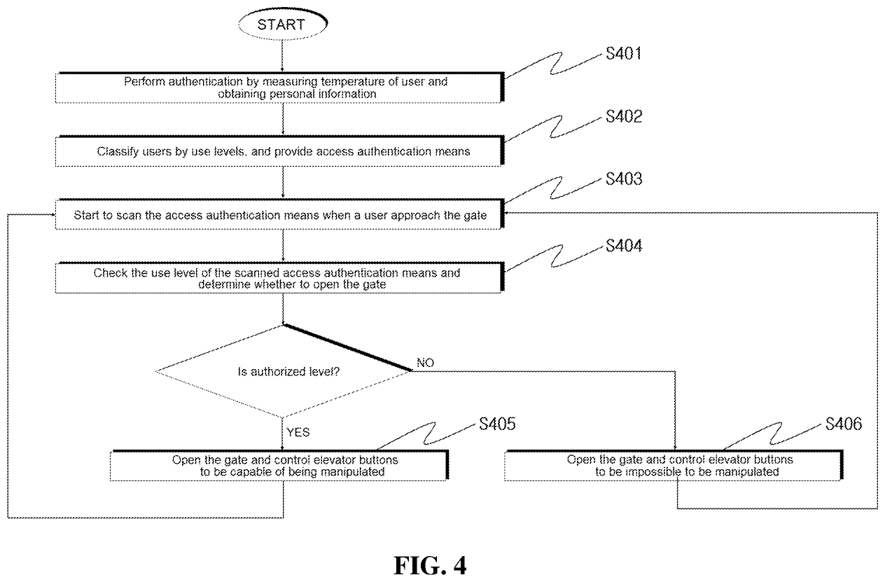

Next, referring to FIG. 4, a process of actively controlling access of hospital users utilizing the hospital access control system 100 will be described in order.

FIG. 4 is a flow chart showing a method of controlling access of users in the hospital utilizing the hospital access control system according to the embodiment of the present invention.

First, the visitor authentication unit 110 measures temperature of hospital users, who pass through the screening center, using a thermal imaging camera or a temperature measuring sensor, and then, obtains and stores personal information from the users through their terminals (S401).

Next, the access authentication means provider classifies the users by the first to fourth access areas based on the personal information of each hospital user, and provides access authentication means allocated by the access areas (S402). In the above process, each user receives an access authentication means appropriate for his or her access purpose, for instance, an RF card, a card or a bracelet on which a character code is printed, registration of biometric information, or others.

Next, when the user approaches a specific gate and scans his or her access authentication means through the access authentication means reader mounted on the gate (S403), the gate control unit determines whether the access area where the user scans his or her access authentication means is an authorized access area to open the corresponding gate and determines whether disinfection is completely carried out, decides whether to open the gate, and outputs a message (a screen display, a voice, a beep sound, or a light) (S404).

If the access area is the authorized access area to open the corresponding gate, the gate control unit opens the corresponding gate or controls buttons of the corresponding elevator to be capable of being manipulated (S405). On the contrary, if the access area is not the authorized access area to open the corresponding gate, the gate control unit disapprove opening of the corresponding gate and outputs a corresponsive message (a screen display, a voice, a beep sound, or a light) so that the user can realize it (S406). Meanwhile, all of the steps S401 to S406 are recorded automatically and stored in the database.

As described above, while the present invention has been particularly shown and described with reference to the preferred embodiment thereof, it will be understood by those of ordinary skill in the art that various changes, modifications and equivalents may be made in the present invention without departing from the technical scope and idea of the present invention.

1. A hospital access control system comprising:
a visitor authentication unit for confirming hospital users and obtaining personal information;
an access authentication means provider which subdivides and classifies the users by predetermined access areas based on the personal information provided from the visitor authentication unit, and gives access authentication means allotted by the access areas to the classified users; and
a gate control unit which is connected with each gate in the hospital and with an access authentication means reader disposed at each gate, determines whether the access area where the user scanned his or her access authentication means through a specific access authentication means reader is an authorized access area when the user scans his or her access authentication means, and determines whether to open the gate.

2. The hospital access control system according to claim 1, wherein the visitor authentication unit comprises:
a temperature measuring means for measuring temperature of the user using a thermal imaging camera or a temperature measuring sensor; and
a personal information obtaining means for obtaining personal information from the user through a terminal.

3. The hospital access control system according to claim 1, wherein the access authentication means provider classifies hospital staff or cooperative firm workers to a first access area, patients and protectors to a second access area, visitors to a third access area, and other users to a fourth access area.

4. The hospital access control system according to claim 3, wherein the access authentication means include character codes including RF cards, barcodes or QR codes, and mobile cards, and biometric information,
wherein the access authentication means reader includes an RF card reader, a character code scanner, and biometric information scanner for recognizing biometric information, such as fingerprint, vein or iris, and
wherein the gate control unit checks whether the access area where the user scans his or her access authentication means corresponds to any one among the first to fourth access areas based on any one of the RF card, the character code and the biometric information scanned through the access authentication means reader.

5. The hospital access control system according to claim 1, wherein the gate control unit differentially applies locations and opening hours of gates, which are authorized to be opened, according to the access areas.

6. The hospital access control system according to claim 1, wherein the access authentication means further includes a database for automatically recording and storing the locations and scanning time of the access authentication means readers where the access authentication means is scanned.

7. The hospital access control system according to claim 1, wherein the gate control unit is linked with a firefighting system constructed in the hospital so as to forcibly open all gates in the hospital in case of fire.

8. The hospital access control system according to claim 1, wherein the gate control unit is connected with all elevators in the hospital to control buttons of the elevators to be capable of being manipulated if the access area where the access authentication means is scanned through the access authentication means reader in the elevator is the authorized access area.

9. The hospital access control system according to claim 1, wherein the hospital access control system is linked with an electronic medical record system (EMR) or an order communication system (OCS) constructed in the hospital.

10. The hospital access control system according to claim 4, wherein the gate control unit synchronizes each of the access authentication means readers mounted at each of the gates in the hospital and the present conditions of each access area in real time.

11. A hospital access control method using a hospital access control system, comprising the steps of:
confirming hospital users through a visitor authentication unit and obtaining personal information;
classifying the hospital users by predetermined access areas using an access authentication means provider, based on the personal information of each user obtained through the visitor authentication unit, and giving access authentication means allotted by the access areas to the classified users; and
determining, through a gate control unit connected with each gate and an access authentication means reader disposed at each gate, whether the access area where the access authentication means is scanned is the authorized access area when the access authentication means is scanned through the access authentication means reader so as to decide whether to open the gate.
